(19) United States
(12) Patent Application Publication
(10) Pub. No.: US 2005/0284850 A1
(43) Pub. Date: Dec. 29, 2005

(54) Method and apparatus for automated assembly and laser welding of medical devices

(75) Inventor: Steven K. Boyd, Litchfield Park, AZ (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/876,303

(22) Filed: Jun. 24, 2004

(51) Int. Cl. B23K 26/00; A61N 1/00

(52) U.S. Cl. 219/121.63; 607/122; 607/36

(57) ABSTRACT

A method and system for assembling a component within a medical device that includes a test station to determine whether the component is in a predetermined working state, and an orientation sensor to sense an orientation vector of the component. A first sensor senses a position of the component within an aperture of the device and plots a weld path associated with the component. An installation head obtains the component, advances the component between the test station, the orientation sensor and the first sensor, and through the aperture, and a weld head generates a weld along a generated weld path.

### FIELD OF THE INVENTION

The present invention relates generally to medical devices; and, more particularly, to the assembly and laser welding of components in a medical device.

### BACKGROUND OF THE INVENTION

Certain medical devices typically have a metal case and a connector block mounted to the metal case. The connector block includes receptacles for leads used for electrical stimulation and/or sensing of physiological signals. A battery and circuitry associated with the particular medical device, e.g., pacemaker circuitry, defibrillator circuitry, etc., is hermetically sealed within the case. Electrical feedthroughs are employed to connect the leads outside the metal case with the medical device circuitry and the battery inside the metal case.

Electrical feedthroughs serve the purpose of providing an electrical circuit path extending from the interior of the hermetically sealed metal case to an external point outside the case while maintaining the hermetic seal of the case. A conductive path is provided through the feedthrough by a conductive pin, which is electrically insulated from the case itself. Such feedthroughs typically include a ferrule which permits attachment of the feedthrough to the case, the conductive pin, and a hermetic glass or ceramic seal which supports the pin within the ferrule and isolates the pin from the metal case. For example, illustrative feedthroughs are shown in U.S. Pat. No. 4,678,868 issued to Kraska, et al. and entitled “Hermetic electrical feedthrough assembly,” in which an alumina insulator provides hermetic sealing and electrical isolation of a niobium conductor pin from a metal case. Further, for example, a filtered feedthrough assembly for implantable medical devices is also shown in U.S. Pat. No. 5,735,884 issued to Thompson, et al. and entitled “Filtered Feedthrough Assembly For Implantable Medical Device,” in which protection from electrical interference is provided using capacitors and zener diodes incorporated into a feedthrough assembly.

Recent advances have enable feedthrough components to be reduced in size to a range of approximately 0.070 inches in diameter, with further size reductions expected in the future. As a result of the microscopic nature of the components, there is a need for manufacturing assemblies associated with the assembly and hermetic welding of implantable medical devices to be more fully automated to maintain quality and reasonable cycle times.

### BRIEF DESCRIPTION OF THE DRAWINGS

Aspects of the present invention will be readily appreciated as they become better understood by reference to the following detailed description when considered in connection with the accompanying drawings, wherein:

FIG. 1A is a schematic bottom view of an exemplary medical device having one or more components assembled utilizing the method and apparatus according to the present invention;

FIG. 1B is a side view of the device of FIG. 1;

FIG. 1C is a top view of the device of FIG. 1;

FIG. 2 is a sectional view of an exemplary feedthrough assembled utilizing the laser welding technique according to the present invention;

FIG. 3 is a schematic diagram of an automated assembly system according to the present invention;

FIG. 4 is a schematic diagram of a portion of an installation station of a vision controlled laser welding portion included in an automated assembly system according to the present invention;

FIG. 5A is a front view of an installation head of the installation station of FIG. 4;

FIG. 5B is a side view of an installation head of the installation station of FIG. 4;

FIG. 5C is a cross-sectional view of the installation head, taken along section line AA of FIG. 5A;

FIG. 5D is a cross-sectional view of the installation head, taken along section line BB of FIG. 5A;

FIG. 6 is a schematic diagram of a tool change station of the installation station of FIG. 5;

FIG. 7 is a schematic diagram of a part presentation assembly of the installation station of FIG. 4;

FIG. 8 is a schematic diagram of a test station included in an installation station of a vision controlled laser welding portion included in an automated assembly system according to the present invention;

FIG. 9 is a schematic diagram of a component positioned over an orientation sensing device in an automated assembly system according to the present invention;

FIG. 10 is a schematic diagram of a welding station of a vision controlled laser welding portion included in an automated assembly system according to the present invention;

FIG. 10A is a partially expanded view of an upper portion of the welding station of FIG. 10;

FIG. 10B is a schematic diagram of a lower portion of the welding station of FIG. 10;

FIG. 10C is a schematic diagram of a sealing pad of an automated assembly system according to the present invention;

FIG. 10D is a cross sectional view of a lower portion of the welding station of FIG. 10; and

FIG. 11 is a flowchart of a method for assembling a component within a medical device according to the present invention.

### DETAILED DESCRIPTION OF THE DRAWINGS

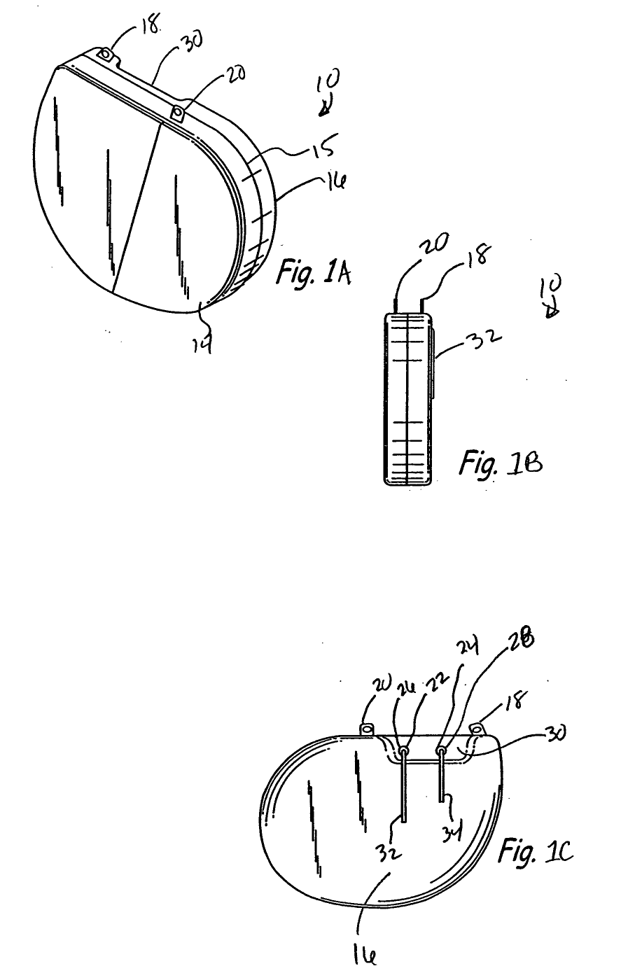

FIG. 1A is a schematic bottom view of an exemplary medical device having one or more components assembled utilizing the method and apparatus according to the present invention. FIG. 1B is a side view of the device of FIG. 1. FIG. 1C is a top view of the device of FIG. 1. As illustrated in FIGS. 1A-C, a medical device 10 that can include components that are assembled and welded using the method and apparatus of the present invention may take the form of an implantable pacemaker that includes at least one or both of pacing and sensing leads (not shown) to sense electrical signals attendant to cardiac depolarization and repolarization and to provide pacing pulses for causing depolarization of cardiac tissue in the vicinity of the distal ends thereof. For example, medical device 10 may be an implantable cardiac pacemaker such as that described in U.S. Pat. No. 5,158,078 to Bennett et al.; U.S. Pat. No. 5,312,453 to Shelton et al.; or U.S. Pat. No. 5,144,949 to Olson et al., hereby incorporated herein by reference in their respective entireties.

Medical device 10 may also be an implantable pacemaker-cardioverter-defibrillator (PCD) corresponding to any of the various commercially-available implantable PCDs. For example, the present invention may be practiced in conjunction with PCDs such as those described in U.S. Pat. No. 5,545,186 to Olson et al.; U.S. Pat. No. 5,354,316 to Keimel; U.S. Pat. No. 5,314,430 to Bardy; U.S. Pat. No. 5,131,388 to Pless; or U.S. Pat. No. 4,821,723 to Baker, et al., all hereby incorporated herein by reference in their respective entireties.

Alternatively, medical device 10 may be an implantable neurostimulator or muscle stimulator such as that disclosed in U.S. Pat. No. 5,199,428 to Obel et al.; U.S. Pat. No. 5,207,218 to Carpentier et al.; or U.S. Pat. No. 5,330,507 to Schwartz, or an implantable monitoring device such as that disclosed in U.S. Pat. No. 5,331,966 to Bennett et al., all of which are hereby incorporated by reference herein in their respective entireties.

Further, for example, medical device 10 may be a defibrillator, an implantable cardioverter/defibrillator (ICD), a brain stimulator, a gastric stimulator, a drug pump, or any other medical device having one or more components assembled utilizing the method and apparatus according to the present invention. Therefore, the present invention is believed to find wide application in any form of medical electrical device.

In the example where medical device 10 is an implantable cardiac device, device 10 includes a first shield 14 and a second shield 16 that are joined together by a weld formed along a seam 15 following placement of the internal components within shields 14, 16 to seal device 10. Together, shield 14 and shield 16 define an enclosure for the internal components of device 10. In addition, one or more fasteners 18 and 20 may be mounted on the exterior of device 10 for fixation of the device within the implanted environment. Shields 14 and 16 and fasteners 18 and 20 may be formed from titanium, for example.

Feedthrough assemblies 22 and 24 are positioned within apertures 26 and 28, respectively, located along an indented portion 30 of shield 16. A number of electrically conductive pins 32 and 34 extend outward from feedthrough assemblies 22 and 24, respectively. The interface between electrically conductive pins 32 and 34 and the interior components of device 10 is hermetically sealed to protect the components from the implanted environment. In this way, feedthrough assemblies 22 and 24 are used to connect any desired number and type of conductors from the exterior of the device 10 to the interior thereof. Although two feedthrough assemblies 22 and 24 are illustrated in FIG. 1C, the medical device 10 could include any desired number of feedthrough assemblies, depending on the number of conductors included with the device.

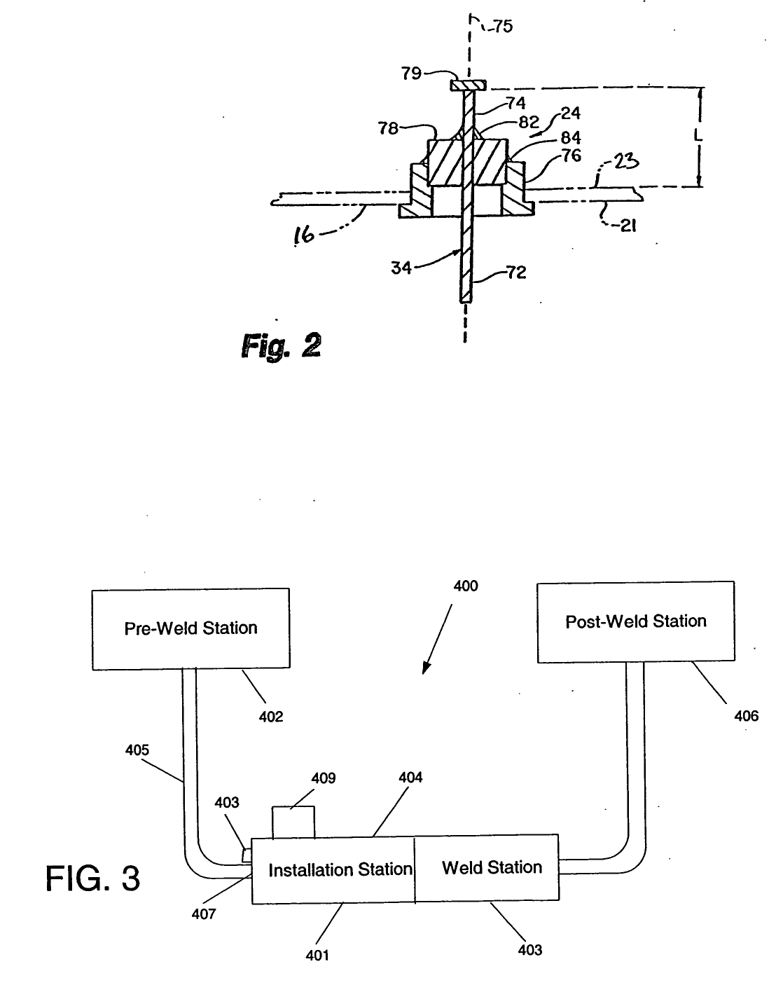

FIG. 2 is a sectional view of an exemplary feedthrough assembled utilizing the laser welding technique according to the present invention. It will be recognized that method and apparatus for assembling a medical device according to the present invention may be utilized to assemble any feedthrough assembly or other component of the medical devices, and therefore is not intended to be limited to use in assembling the feedthrough assembly illustrated in FIG. 2. As illustrated in FIG. 2, feedthrough assembly 24, for example, includes a ferrule 76 disposed around an electrically conductive pin 34 supported by an insulator 78 and having a longitudinal axis 75 extending therethrough. The insulator 78 is secured to the ferrule 76 by means of a braised joint 84. Similarly, the electrically conductive pin 34 is secured to the insulator 78 by way of a braised joint 82.

Shield 16 includes an exterior surface 21 and an interior surface 23. The feedthrough 24 in FIG. 2 is shown in sealing engagement with one side, i.e., exterior surface 21, of shield 16, formed utilizing the method and apparatus of the present invention, described below in detail. With the feedthrough 24 in sealing engagement with shield 16, a first end 74 of electrically conductive pin 34 projects from interior surface 23 of shield into the interior of shield 16 and may be terminated with a pin termination pad 79, e.g., a Kovar pad. The pin termination pad 79 generally lies perpendicular to the longitudinal axis 75 extending through the pin 34. A second end 72 of electrically conductive pin 34 projects from the exterior surface 21 to the exterior of the shield 16. Generally, the ferrule 76 is sealed to shield 16 by welding formed utilizing the method and apparatus of the present invention, described below in detail.

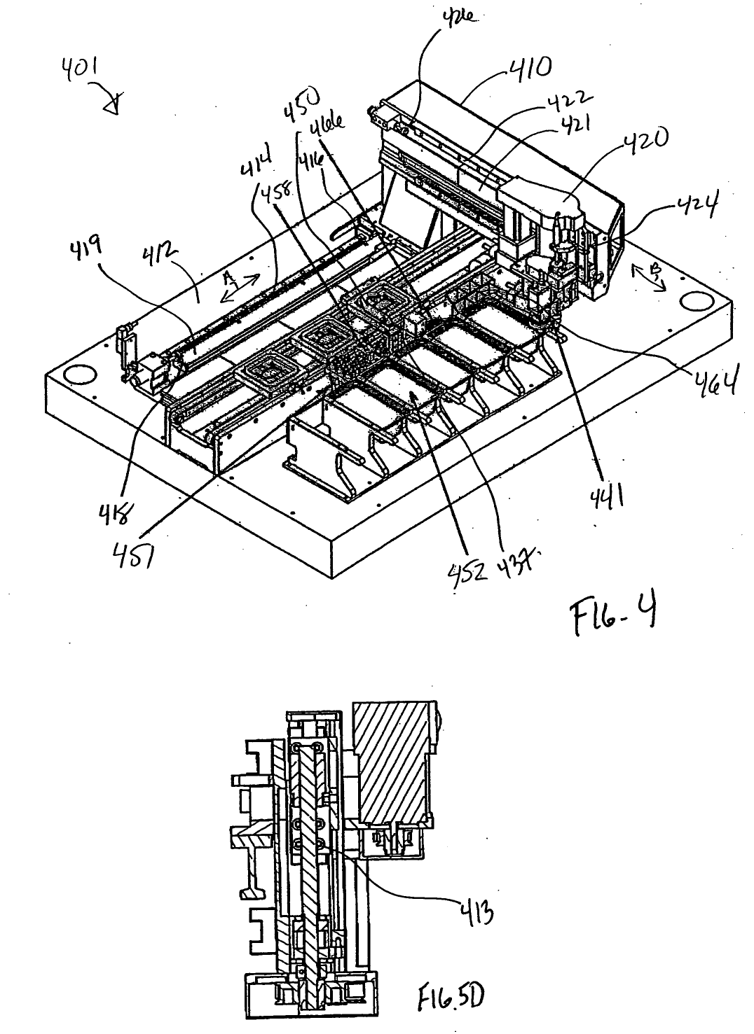

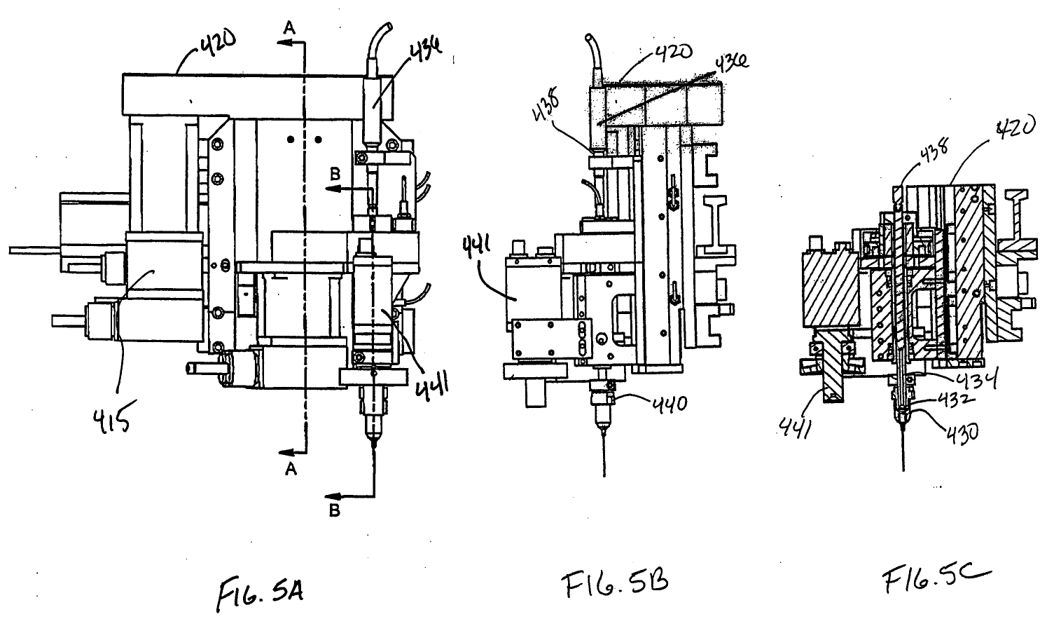

FIG. 3 is a schematic diagram of an automated assembly system according to the present invention. As illustrated in FIG. 3, an automated assembly system 400 according to the present invention includes a user interface having a microprocessor 409 for controlling operation of motors and sensors included in system 400, as will be described in detail below. System 400 includes a pre-weld portion 402 for preparing shields that are subsequently fed into a vision controlled laser welding portion 404. For example, fasteners 18 and 20 are welded to shield 16, or a case block (not shown) for grounding feedthrough 22 and 24 to shield 16 is welded to shield 16 during pre-weld portion 402 of system 400. During a post-weld portion 406, the welded device is inspected and placed in a final condition for shipment from the assembly and later distribution. For example, an insulative layer of conductive pins 32 and 34 is trimmed during post-weld portion 406.

Welding portion 404 includes an installation station 401 and a welding station 403. Once the device, such as shield 16, for example, is prepared in the pre-weld portion 402, the device is advanced along a carrier 405, such as a conveyor belt, and a matrix identification is read by a device identification camera 403 so that the microprocessor is able to determine the device type. The device is further advanced so as to be positioned within installation station 401 via an opening 407. Once the device is positioned within installation station 401, desired components, such as feedthroughs, for example, are retrieved and appropriately positioned on the device, as will be described below. Once the required number of components have been installed on the device, the device is advanced from installation station 401 to welding station 403 along carrier 405. Once positioned within welding station 403, a weld is completed for each component after it is determined that the component is properly positioned on the device, as will be described below.

FIG. 4 is a schematic diagram of a portion of an installation station of a vision controlled laser welding portion included in an automated assembly system according to the present invention. As illustrated in FIG. 4, installation station 401 includes an arm 410 that is capable of being advanced on a base platform 412 along a y-axis, indicated by arrow A, via rails 414 positioned within base platform 412. Arm 410, which is shown in FIG. 4 positioned at a first end 416 of rails 414, is automatically advanced to a determined position along rails 414 between the first end 416 and a second end 418 of rails 414 by being driven by a linear motor 419 located on base platform 412. Similarly, an installation head 420 is capable of being advanced relative to the base platform 412 along an x-axis, indicated by arrow B, via rails 422 positioned within arm 410. Installation head 420, which is shown in FIG. 4 positioned at a first end 424 of rails 422, is automatically advanced to a determined position along rails 422 between the first end 424 and a second end 426 of rails 422 by being driven by a linear motor 421 located on arm 410.

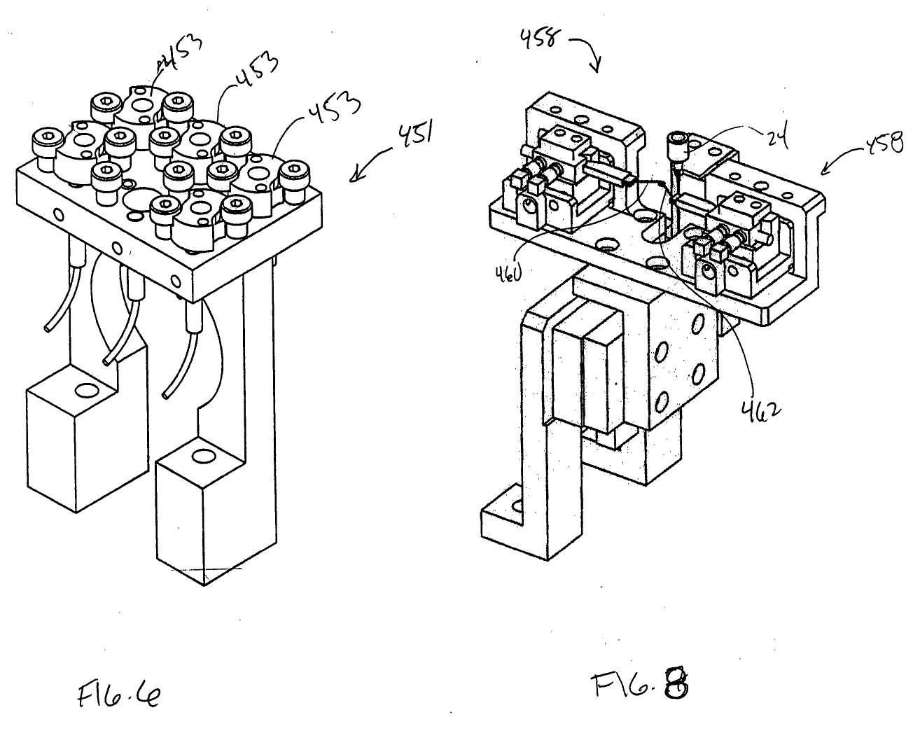

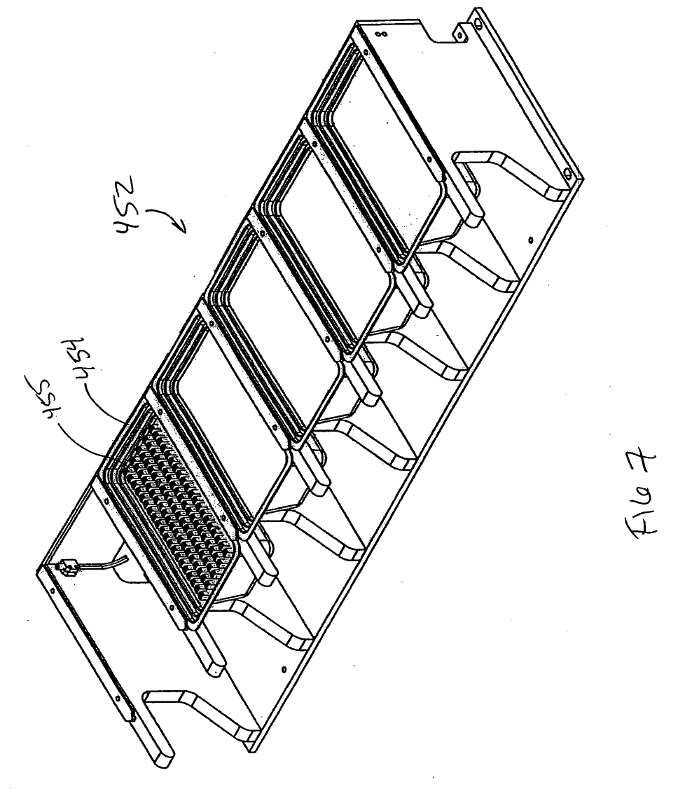

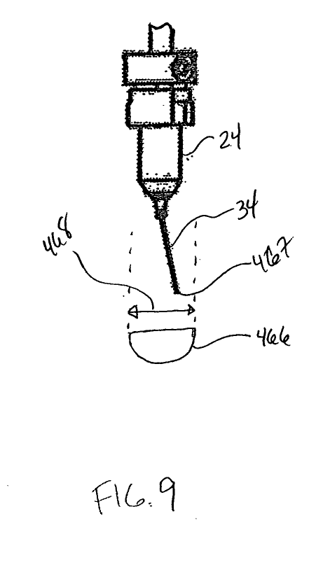

FIG. 5A is a front view of an installation head of the installation station of FIG. 4. FIG. 5B is a side view of an installation head of the installation station of FIG. 4. FIG. 5C is a cross-sectional view of the installation head, taken along section line M of FIG. 5A. FIG. 5D is a cross-sectional view of the installation head, taken along section line BB of FIG. 5A. As illustrated in FIG. 5D, installation head 420 is automatically advanced in the z-direction relative to base platform 412 by a ball screw arrangement 413 included in installation head 420 so that insulation head can be raised or lowered to a desired distance relative to base platform 412 via a motor 415, as will be described below.

As illustrated in FIGS. 5A-5C, installation head 420 includes a vacuum nozzle 430 positioned at one end 432 of a shaft 434, which can be automatically advanced in the z-direction via ball screw arrangement 413, and a pressure sensor 436 positioned at the other end 438 of shaft 434. A motor 440 positioned along vacuum nozzle 430 enables vacuum nozzle 430 to be automatically rotated about shaft 434. Sensor 436 senses vertical pressure at vacuum nozzle 430 indicating the component is fully installed within the desired position along the device, as will be described below. Finally, a sensor 441, such as a camera, for example, is positioned above and offset from vacuum nozzle 430 to locate the desired position for placing the component on the device, as will be described below.

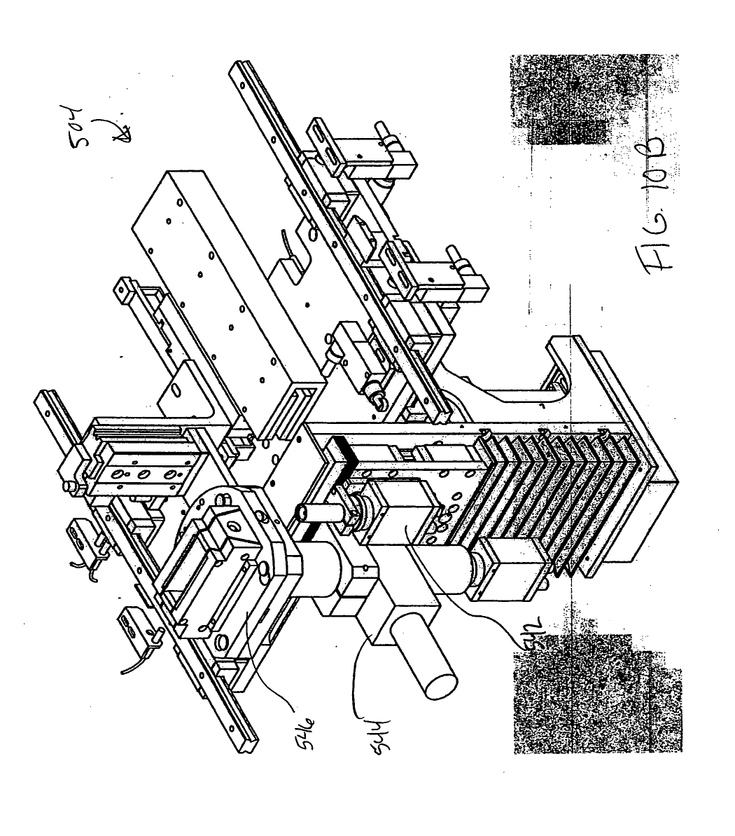

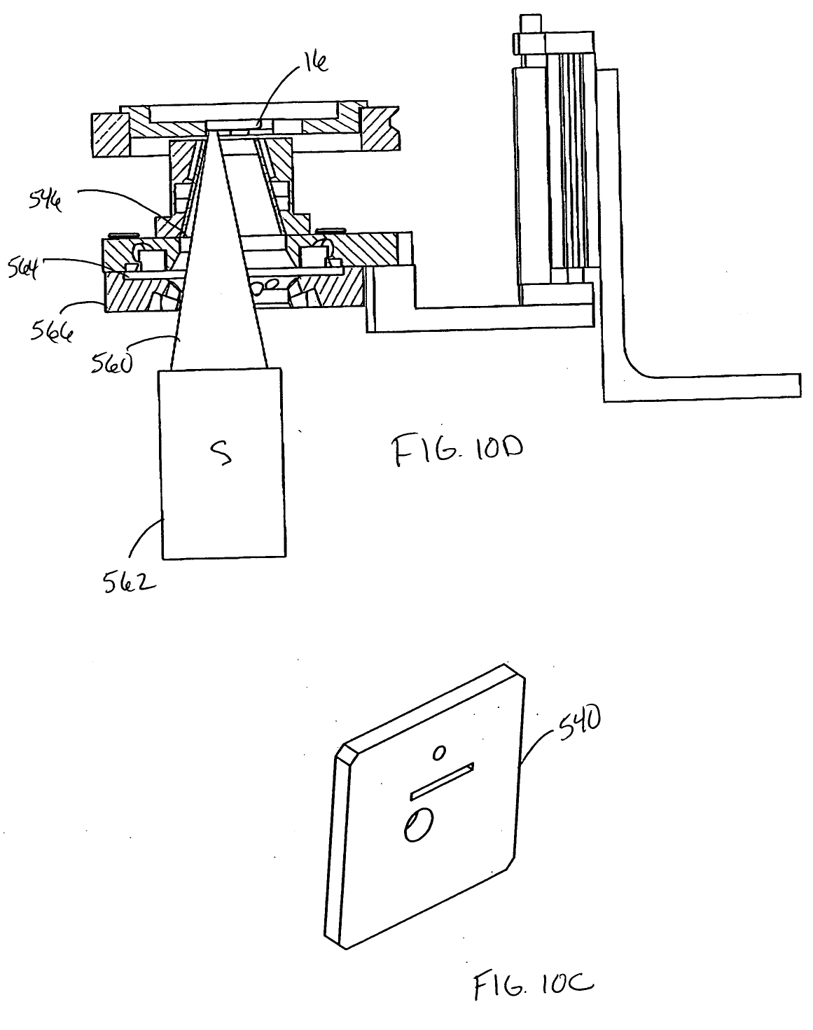

Returning to FIG. 4, the device is advance within installation station 401 until a locating pin 437 aligns with a pin aperture 439 on device 16 and is advanced within aperture 439, similar to an arrangement illustrated in FIG. 10A and described below. A clamp 450 rotates downward and engages against device 16 to prevent movement of device 16 during the assembly function. Once device 16 is fixedly positioned by clamp 450, installation head 420 is advanced, using the combined movement of arm 410 relative to base platform 412 along the y-axis and installation head 420 relative to arm 410 along the x-axis and the z-axis, to a tool change station 451 that includes various differing nozzles 453, as shown in FIG. 6. A nozzle corresponding to the component associated with the identified device, such as nozzle 430, is located and centrally positioned on installation head 420.

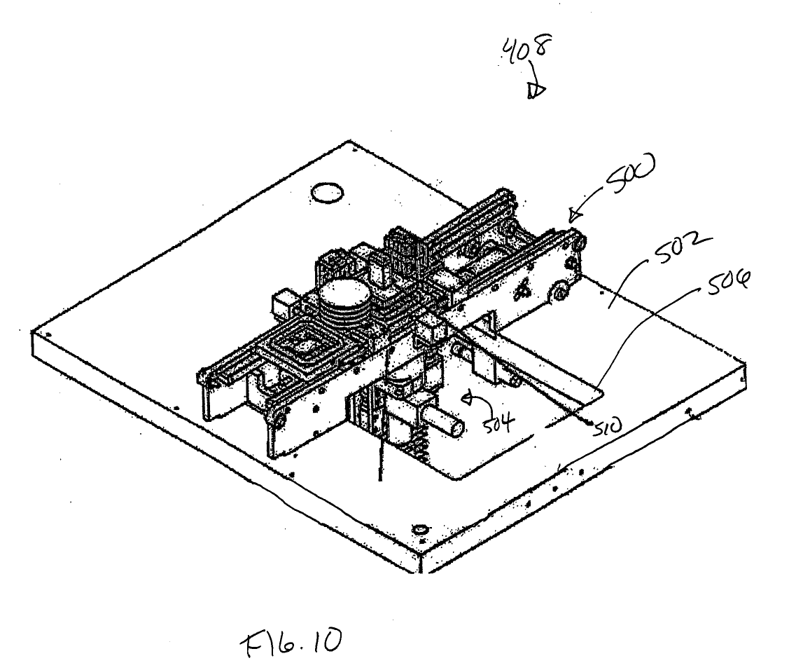

FIG. 7 is a schematic diagram of a part presentation assembly of the installation station of FIG. 4. Once positioned on installation head 420, nozzle 430 is positioned over a proper tray included in a part presentation assembly 452 corresponding to the component associated with the device. For example, installation head 420 advances over tray 454 until sensor 441 locates a component 455 stored on tray 454. A determination is then made as to whether a pattern associated with the component 455 corresponds with the pattern expected for that component. If the pattern of component 455 is corresponds with the expected pattern, installation head 420 is advanced so that, based on the known offset of sensor 441 from nozzle 430, nozzle 430 is centered over the component 455. Installation head 420 is then advanced so that nozzle 430 is lowered over the located component 455, and a vacuum is applied so that the component 455 is picked up by nozzle 430. Once the vacuum is determined to be present, indicating the component has been picked up, installation head 430 is advanced so that the component is positioned within a test station 458.

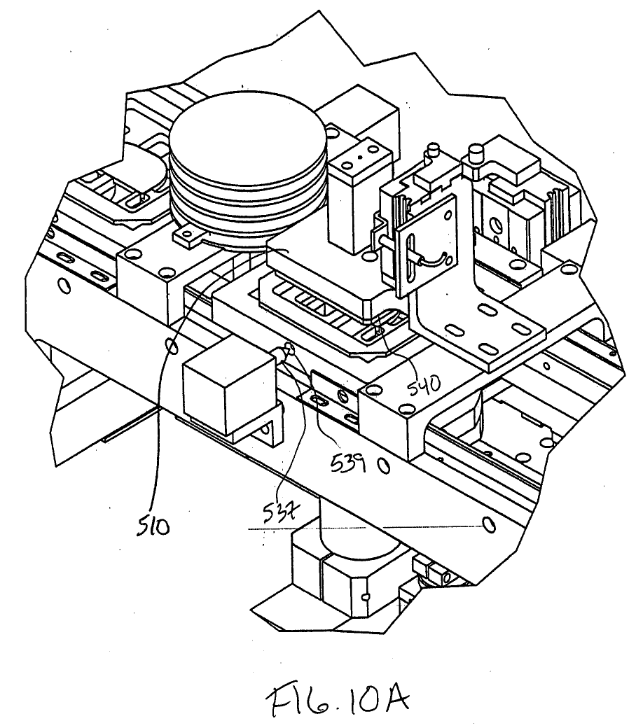

FIG. 8 is a schematic diagram of a test station included in an installation station of a vision controlled laser welding portion included in an automated assembly system according to the present invention. As illustrated in FIG. 8, test station 458 includes a pair of probes 460 and 462, so that once feedthrough 24 is positioned in test station 458 by installation head 420, probes 460 and 462 are positioned against feedthrough 24 in order to test the capacitance of the feedthrough. If the capacitance is determined to be outside a predetermined range, installation head 420 deposits the component in a bin of a number of bins 464 that corresponds to tray 454 associated with the selected component. If the capacitance is within the predetermined range and therefore the component is in proper condition, installation head 420 advances the component over a component orientation sensing device 466, such as an upward vision camera, for example.

FIG. 9 is a schematic diagram of a component positioned over an orientation sensing device in an automated assembly system according to the present invention. As illustrated in FIG. 9, once installation head 420 advances feedthrough 24 to be centrally located over orientation sensing device 466, a determination is made as to whether a distal tip 467 of conductive pin 34 is oriented such that tip 467 is located within a sensing range 468 associated with orientation sensing device 466. If tip 467 is not located within sensing range 468, installation head 420 deposits the component in the bin of the bins 464 that corresponds to tray 454 associated with the selected component. If tip 467 is located, installation head 420 is advanced so that sensor 441 is positioned over the device to determine the location of the aperture in which the component is to be assembled. The location of the aperture is known based on the matrix identification previously obtained by identification camera 403.

Once the aperture is located, installation head 420 advances the component over the aperture, centrally locating tip 467 over the aperture. Installation head 420 is then lowered along the z-axis towards the aperture to a point where tip 467 is positioned inside aperture 467 a distance corresponding to the thickness of shield 16. Once tip 467 properly positioned within aperture 467, installation head 420 continues to be lowered along the z-axis, vectoring conductive pin 34 through the aperture using the x and y-axis to compensate for any lead out of perpendicularity of conductive pin 34 relative to nozzle 430, as determined by orientation device 466. Installation head 420 continues to be lowered in the z-direction until pressure is sensed by sensor 436, signifying that the component is fully seated within the device.

The assembly process is repeated until the proper number of components (feedthroughs 22 and 24, for example) are assembled within the device. The device is then released by clamp 450, and carrier 405 advances the device within welding station 408.

FIG. 10 is a schematic diagram of a welding station of a vision controlled laser welding portion included in an automated assembly system according to the present invention. FIG. 10A is a partially expanded view of an upper portion of the welding station of FIG. 10. FIG. 10B is a schematic diagram of a lower portion of the welding station of FIG. 10. Welding station 408 includes a top portion 500 positioned on a platform base 502, and a bottom portion 504 extending upward towards top portion 500 through a cutout portion 506 formed in platform base 502. Device 16 is advance from installation station 401 into welding station 408 until a locating pin 537 of welding station 408 aligns with a pin aperture 539 on device 16 and is advanced within aperture 539. A clamp 510 rotates downward and engages against device 16 to prevent movement of device 16 during the welding process. Clamp 510 includes a sealing pad 540, an example of which is illustrated in FIG. 10C. Pad 540, which is formed from of a silicone foam material, for example, fixedly positions the components in the device in order to prevent movement of components during the welding process, and seals the upper portion of the device during the welding process.

A sensor 542, such as a camera, for example, included in bottom portion 504, is then positioned under one of the apertures in the device in which a component has been positioned within the device by installation station 401. The microprocessor then compares the diameter of the aperture and the diameter of the component to determine whether the component is properly positioned within the aperture. Once a determination is made for each of the components, the microprocessor plots a weld path for each of the components using the image generated by sensor 542. A weld head 544 is positioned under the device so that weld head 544 and sealing pad of 540 of clamp 510 form a gas suite 546. An inert gas, such as argon for example, is then injected into the gas suite 546 so that a pocket of heavier than air inert gas is formed along the weld area on the device during the weld operation.

FIG. 10D is a cross sectional view of the lower portion of the welding station of FIG. 10. As illustrated in FIG. 10D, during generation of the weld, a laser beam 560 is introduced to device 16 via a laser nozzle 562 through a protection glass 564 positioned within a light unit 566, and the inert gas is introduced via an air cylinder 568.

Once components that were determined to be properly positioned are welded into the device, and if there were components determined not to be properly positioned, clamp 510 is raised and locating pin 537 is retracted from pin aperture 539 and repositioned within pin aperture 539 in a single motion. Because of the taper that is located at the distal tip of locating pin 537, the single motion of retracting locating pin 537 and inserting relocating pin 537 within pin aperture 539 has the effect of shaking the device so that the non-welded component or components are adjusted to be properly positioned within the aperture.

Once the retraction and repositioning of locating pin 537 within pin aperture 539 has been performed, clamp 510 is positioned on the device as previously described, sensor 542 is repositioned under the device, and a determination is made for each non-welded component as to whether the component is properly positioned with the aperture. Weld paths are plotted for the properly positioned components and the weld is then formed, as described above. This process is repeated a predetermined number of times, such as three for example, and if components remain unwelded after the process has been performed the predetermined number of times, the device is rejected. Once all of the components have been welded, clamp 510 is removed, and locating pin 537 is retracted from pin aperture 539 so that the device is transferred along carrier 405 to post-weld station 406.

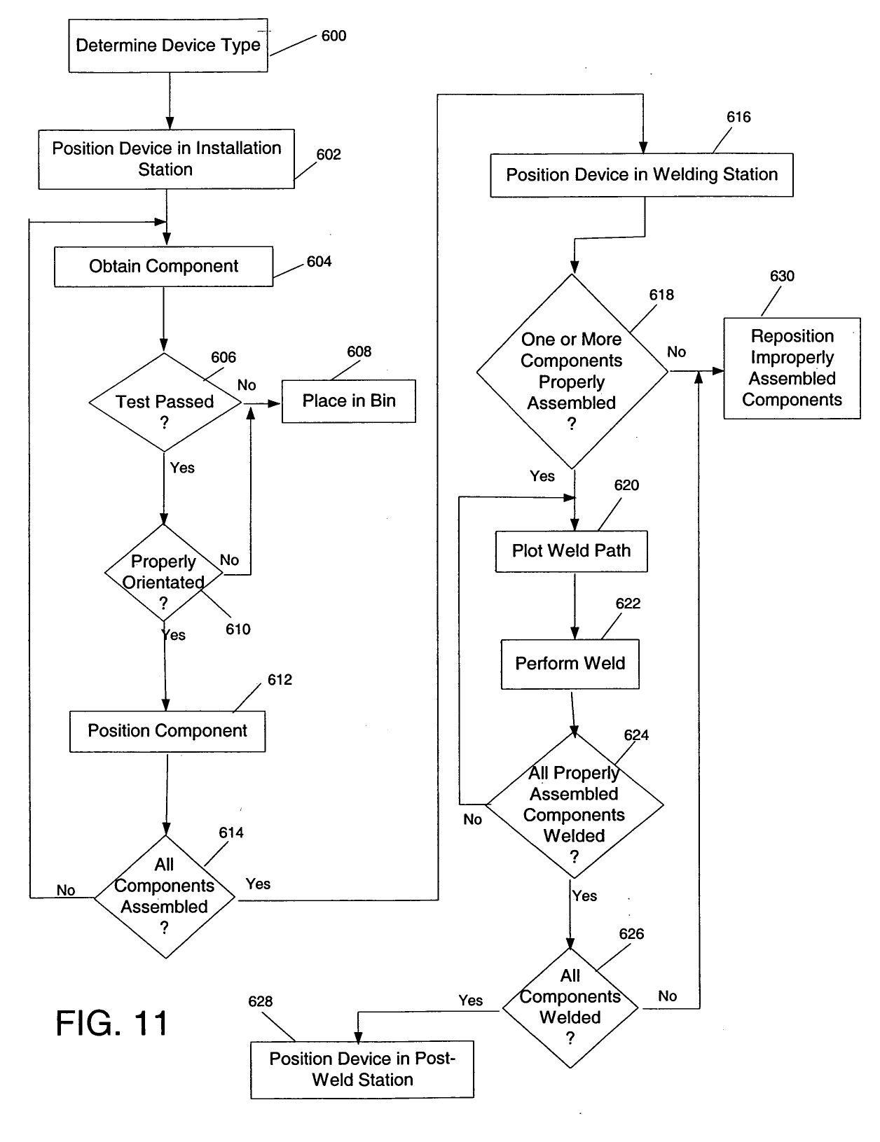

FIG. 11 is a flowchart of a method for assembling a component within a medical device according to the present invention. As illustrated in FIG. 11, a method of assembling and laser welding components in a medical device according to the present invention includes determining the type of device, block 600, and positioning the device within the installation station, Block 602. The installation head is then advanced to obtain a component associated with the determined type of device, block 604, and advances the component to the test station to determine whether the component is in a proper working condition. If the component is not in proper working condition, the installation head places the component in the proper rejection bin associated with that component, block 608.

If the component is determined to be in proper operating condition, the component is advanced over an orientation sensor to determine whether the component is properly orientated, block 610. If not properly orientated, the installation head places the component in the proper rejection bin associated with that component, block 608. If the component is properly oriented, the component is positioned with an aperture on the device, block 612, a determination is made as to whether all of the components associated with assembling the device have been assembled within the device, block 614.

Once all components have been assembled within the device, the device is transferred from the installation station to the welding station, block 616, and a determination is made as to at least one component is properly assembled within the device, block 618. If one or more of the components is properly assembled within the device, a weld path is plotted and the weld. is performed for each of the properly assembled components, blocks 620-624. Once each of the properly assembled components has been welded, a determination is made as to whether all of the required components for the determined device have been welded, block 626.

If all of the required components associated with the identified device have been welded, the device is advanced to the post-welding station. If all of the components have not yet been welded, an attempt is made to adjust the non-welded components, block 630, so that the non-welded components are properly assembled within the device, and the welding process, blocks 618-626 is repeated. This readjustment process can be repeated a predetermined numbers of times, such as three times as described above, so that if all of the components are not welded after the predetermine number of attempts, the device is rejected.

Some of the techniques described above may be embodied as a computer-readable medium comprising instructions for a programmable processor such as microprocessor 409. The programmable processor may include one or more individual processors, which may act independently or in concert. A “computer-readable medium” includes but is not limited to any type of computer memory such as floppy disks, conventional hard disks, CR-ROMS, Flash ROMS, nonvolatile ROMS, RAM and a magnetic or optical storage medium. The medium may include instructions for causing a processor to perform any of the features described above for initiating a session of the escape rate variation according to the present invention.

The preceding specific embodiments are illustrative of the practice of the invention. It is to be understood, therefore, that other expedients known to those of skill in the art or disclosed herein may be employed without departing from the invention or the scope of the appended claim. It is therefore to be understood that the invention may be practiced otherwise than as specifically described, without departing from the scope of the present invention. As to every element, it may be replaced by any one of infinite equivalent alternatives, only some of which are disclosed in the specification.

1. A system for assembling a component within a medical device, comprising:
a test station to determine whether the component is in a predetermined working state;
an orientation sensor to sense an orientation vector of the component;
a first sensor to sense a position of the component within an aperture of the device and to plot a weld path associated with the component;
an installation head to obtain the component, advance the component between the test station, the orientation sensor and the first sensor, and to advance the component through the aperture; and
a weld head generating a weld along the weld path.

2. The system of claim 1, further comprising a microprocessor to determine, in response to the sensed orientation vector, whether a distal tip of the component is within a predetermined range.

3. The system of claim 1, further comprising a microprocessor to determine a lead out of perpendicularity associated with the component in response to the sensed orientation vector, to control the installation head to position a distal tip of the component within the aperture a distance associated with a thickness of the device, and to vector the component through the aperture to compensate for the determined lead out of perpendicularity of the component.

4. The system of claim 1, further comprising:
a nozzle positioned on the installation head to form a vacuum to fixedly engage the installation head and the component; and
a second sensor to sense contact between the nozzle and the device as the installation head advances the component through the aperture.

5. The system of claim 1, further comprising:
a second sensor to sense, subsequent to the component being advanced through the aperture by the installation head, a position of the component within the aperture; and
a microprocessor to compare, in response to the sensed position of the component, a diameter of the aperture with a diameter of the component.

6. The system of claim 1, further comprising a clamp having a seal member to fixedly position the component within the device subsequent to the component being advanced through the aperture by the installation head and to form a seal along an upper portion of the device, the weld head capable of being positioned under the device, the seal and the weld head forming a gas suite along the aperture.

7. The system of claim 1, further comprising:
a base platform;
an arm capable of being advance on the base platform to be positioned along a y-axis, the installation head mechanically coupled to the arm and capable of being advanced along the arm to be positioned along an x-axis and a z-axis;
a shaft positioned within the installation head;
a nozzle positioned on the shaft and capable of being rotated about the shaft, the nozzle forming a vacuum to fixedly engage the installation head and the component; and
a second sensor to sense contact between the nozzle and the device as the installation head advances the component through the aperture.

8. The system of claim 1, wherein the component is a feedthrough.

9. A system for assembling a component within a medical device, comprising:
a test station to determine whether the component is in a predetermined working state;
an orientation sensor to sense an orientation vector of the component;
a first sensor to sense a position of the component within an aperture of the device and to plot a weld path associated with the component;
an installation head to obtain the component, advance the component between the test station, the orientation sensor and the first sensor, and to advance the component through the aperture;
a weld head generating a weld along the weld path; and a microprocessor to determine, in response to the sensed orientation vector, a lead out of perpendicularity associated with the component and whether a distal tip of the component is within a predetermined range, the microprocessor controlling the installation head to position a distal tip of the component within the aperture a distance associated with a thickness of the device, and to vector the component through the aperture to compensate for the determined lead out of perpendicularity of the component.

10. The system of claim 9, further comprising:
a nozzle positioned on the installation head to form a vacuum to fixedly engage the installation head and the component; and
a second sensor to sense contact between the nozzle and the device as the installation head advances the component through the aperture.

11. The system of claim 10, further comprising:
a third sensor to sense, subsequent to the component being advanced through the aperture by the installation head, a position of the component within the aperture; and
a microprocessor to compare, in response to the sensed position of the component, a diameter of the aperture with a diameter of the component.

12. The system of claim 11, further comprising a clamp having a seal member to fixedly position the component within the device subsequent to the component being advanced through the aperture by the installation head and to form a seal along an upper portion of the device, the weld head capable of being positioned under the device, the seal and the weld head forming a gas suite along the aperture.

13. The system of claim 1, further comprising:
a base platform;
an arm capable of being advance on the base platform to be positioned along a y-axis, the installation head mechanically coupled to the arm and capable of being advanced along the arm to be positioned along an x-axis and a z-axis; and
a shaft positioned within the installation head, the nozzle positioned on the shaft and capable of being rotated about the shaft.

14. The system of claim 13, wherein the component is a feedthrough.

15. The system of claim 1, further comprising an identification sensor to determine an identification of the device, wherein the installation head obtains the component in response to the determined identification of the device.

16. A method for assembling a medical device, comprising:
obtaining a component associated with the device;
determining whether the component is in a predetermined working state;
determining whether the component has a predetermined orientation;
positioning the component within an aperture associated with receiving the component;
determining whether the component is properly positioned within the aperture;
plotting a weld path associated with the properly positioned component; and
generating a weld along the weld path.

17. The method of claim 16, wherein determining whether the component has a predetermined orientation comprises:
advancing the component to be centrally located over a sensing device; and
determining whether a distal tip of the component is within a sensing range associated with the sensing device.

18. The method of claim 16, wherein determining whether the component has a predetermined orientation includes determining a lead out of perpendicularity associated with the component, and wherein positioning the component within an aperture associated with receiving the component comprises:
positioning a distal tip of the component within the aperture a distance associated with a thickness of the device; and
vectoring the component through the aperture to compensate for the determined lead out of perpendicularity of the component.

19. The method of claim 18, further comprising determining whether the component is fully seated within the aperture.

20. The method of claim 16, wherein determining whether the component is properly positioned within the aperture comprises comparing a diameter of the aperture with a diameter of the component.

21. The method of claim 16, further comprising:
fixedly positioning the properly positioned component within the device;
forming a seal along an upper portion of the device;
positioning a weld head under the device, the seal and the weld head forming a gas suite along the aperture; and
injecting an inert gas within the gas suite.

22. The method of claim 21, wherein determining whether the component has a predetermined orientation comprises:
advancing the component to be centrally located over a sensing device; and
determining whether a distal tip of the component is within a sensing range associated with the sensing device.

23. The method of claim 21, wherein determining whether the component has a predetermined orientation includes determining a lead out of perpendicularity associated with the component, and wherein positioning the component within an aperture associated with receiving the component comprises:
positioning a distal tip of the component within the aperture a distance associated with a thickness of the device; and
vectoring the component through the aperture to compensate for the determined lead out of perpendicularity of the component.

24. The method of claim 23, further comprising determining whether the component is fully seated within the aperture.

25. The method of claim 21, wherein determining whether the component is properly positioned within the aperture comprises comparing a diameter of the aperture with a diameter of the component.

26. A computer readable medium having computer executable instructions for performing a method comprising:
obtaining a component associated with the device;
determining whether the component is in a predetermined working state;
determining whether the component has a predetermined orientation;
positioning the component within an aperture associated with receiving the component;
determining whether the component is properly positioned within the aperture;
plotting a weld path associated with the properly positioned component; and
generating a weld along the weld path.
